(19) United States
(12) Patent Application Publication
(10) Pub. No.: US 2015/0024077 A1
(43) Pub. Date: Jan. 22, 2015

(54) COMPOSITION FOR TREATING SKIN BARRIER AND REDUCING ACNE

(71) Applicant: JOHNSON & JOHNSON CONSUMER COMPANIES INC., Skillman, NJ (US)

(72) Inventors: Nikoleta Batchvarova, West Windsor, NJ (US); Apostolos Pappas, Bridgewater, NJ (US)

(21) Appl. No.: 14/338,072

(22) Filed: Jul. 22, 2014

Related U.S. Application Data
(63) Continuation-in-part of application No. 13/947,473, filed on Jul. 22, 2013.
Application No. 13/947,473 is a continuation-in-part of application No. 13/947,489, filed on Jul. 22, 2013.

(51) Int. Cl. A61K 36/185; A61K 36/32

(52) U.S. Cl. CPC A61K 36/185; A61K 36/32
USPC 424/776; 424/725

(57) ABSTRACT

A composition for applying to skin suffering from acne to treat the acne, the composition comprising an extract of Malva neglecta. The Malva neglecta increases ceramide production at the area of skin affected by acne to treat and/or improve the acne. Additionally, the composition may include cholesterol.

### CROSS-REFERENCE TO RELATED APPLICATION

This application is a continuation in part of application Ser. No. 13/947,473, filed on Jul. 22, 2013, and application Ser. No. 13/947,489, filed on Jul. 22, 2013, both of which applications are hereby incorporated by reference in the present application as if fully set forth herein.

### FIELD OF THE INVENTION

The present invention relates to compositions for treating the skin barrier and reducing acne comprising plant extracts for use on skin. More specifically, the present invention relates to compositions comprising extracts of Malva neglecta for improving the condition and appearance of the skin, such as by improving skin barrier protection, and, improving appearance and/or inhibiting, reducing, or treating formation of acne on skin.

### BACKGROUND OF THE INVENTION

Malva neglecta is typically designated a “weed.” Native to the “Old World,” it has been naturalized throughout North America. Malva neglecta is native to almost all of Europe, from northern Europe (e.g., Denmark, Ireland, Norway, Sweden, United Kingdom), middle Europe (e.g., Austria, Belgium), Southeastern Europe (e.g., Albania, Bulgaria, Croatia, etc.), to Southwestern Europe (e.g., France, Portugal, Spain). It is also found in Western Asia, the Arabian Peninsula, Northwestern Asia (e.g., Armenia, Ga., Kazakhstan, Uzbekistan, Mongolia) and also in China and the Indian subcontinent. In Africa, it is found mostly in North Africa, such as Algeria and Morocco.

Many Malva species are used in traditional medicinal systems around the world, including Malva neglecta. It has also been commonly used as a food. It has not been commercialized as a trade herb. It is also known by various common names—Common mallow, buttonweed, cheeseplant, cheeseweed, dwarf mallow, and roundleaf mallow. According to Plants For A Future (http://www.pfaf.org/user/default.aspx), the online database for medicinal and edible wild plants, Malva neglecta is described for use as anti-inflammatory, anti-phlogistic, astringent, demulcent, diuretic, emollient, expectorant, laxative, poultice, purgative, and salve.

The uses of Malva neglecta disclosed in Plants For A Future, as well as other known uses of Malva neglecta described in other references, are mostly in ingested forms, except for use as an emollient or salve or poultice. Some traditional literature also describes the use of a poultice for eczema.

Plants or botanicals may be formed into compositions for topical application in a variety of manners. A poultice is a soft moist mass, often heated and medicated, that is spread on cloth over the skin to treat an aching, inflamed, or painful part of the body. It can be used on wounds such as cuts. A decoction involves boiling plant material in water to extract certain chemicals or properties. An infusion is prepared by steeping plant material in hot water (like a tea bag). A solvent-based extraction is made by grinding or macerating plant material in a solvent, typically an organic solvent such as an alcohol, acetone, hexane, or chloroform. Typical traditional methods of forming compositions from plants or botanicals, such as described in Plants For A Future and the other prior art references generally employ poultice or decoction or infusion methods of preparation. In particular, traditional art describes use of Malva neglecta in forms such as a water decoction, after removing insoluble parts of the plant taken orally, as a poultice, or an infusion applied to burns, insect bites, and wounds. By using water, these methods typically extract only the most polar constituents, e.g., tannins.

Topical uses of Malva neglecta reported in the prior art (S. Foster and J A Duke, Medicinal Plants and Herbs, pp. 170-171, New York, Houghton Mifflin Company 2000) are limited to wounds and tumors. However, more common species of Malva genus, e.g., Malva sylvestris, are sometimes also extended to Malva neglecta in the form of decoctions or compresses for treating abscesses, boils, burns, eczema, and insect bites. (E. Launert, The Hamlyn Guide to Edible & Medicinal Plants, p. 50; D. Bown, New Encyclopedia of Herbs and Their Uses, pp. 270-271, New York, DK Publishing, Inc. 2001). Traditional literature also describes the preparation of Malva neglecta as a poultice or decoction for medicinal uses described above. Poultices and decoctions are obtained when raw materials are soaked in water with or without heat and may or may not involve separation of plant materials before application. According to this description of preparing Malva neglecta for therapeutic purposes, it is obvious that the most effective preparation would be such where hydrophilic components, e.g., tannins, are extracted in water, more so in boiling water, such as described in E. Launert, Edible & Medicinal Plants. Tannins are naturally occurring plant polyphenols and are hydrophilic components with astringent taste.

The skin is the largest organ of the body and forms an effective barrier between the organism and the environment preventing invasion of pathogens and fending off chemical and physical assaults, as well as the unregulated loss of water and solutes. The maintenance of a barrier against excessive transcutaneous water loss to the environment is critical to survival of all terrestrial animals. In mammals, this barrier is formed by the anucleate, cornified, outermost layers of the epidermis, collectively known as the stratum corneum. The stratum corneum (SC) is viewed currently as a layer of protein-enriched corneocytes embedded in a lipid-enriched, intercellular matrix, the so-called bricks and mortar model. The “bricks” are corneocytes surrounded by a cornified cell envelope made up of proteins, mainly loricrin, filaggrin, and involucrin, and covalently bound to the hydroxyceramide molecules of a lipid envelope. These “bricks” are embedded in a “mortar” of lipid bilayers. The so-called mortar contains a variety of intercellular lipids including, ceramides, free sterols, cholesterolsulphate, and free fatty acids.

As noted in U.S. Pat. No. 5,643,899 to Elias (“Elias”), the intercellular, lamellar, and bilayer sheets of stratum corneum lipids are the key constituents for a functional skin barrier. Elias refers to the three dominant epidermal lipids by weight as ceramides (40%), free fatty acids (20-25%) and cholesterol (20-25%). According to current theory, any disturbances in the epidermal barrier results in a variety of diseases and conditions of the skin and mucous membrane, such as contact dermatitis, ichthyosis, psoriasis, and atopic dermatitis. Recently, skin barrier impairment has been linked to acne vulgaris. Yamamoto et al. (“Impaired water barrier function in acne vulgaris”, Arch Dermatol Res (1995) 287: 214-218) describes significantly lower levels of sphingolipids (ceramides and free sphingosine) in acne patients and also a reduced water barrier function. Yamamoto have observed that the low ceramide levels corresponding to impaired water skin barrier function might lead to the formation of acne or comedones. However, Yamamoto et al. only refer to ceramides and not any other lipid classes, such as free fatty acids. Based on such observations, Thiboutot D., Del Rosso, J. Q. (2013), Acne Vulgaris and the Epidermal Barrier, SKIN STRUCTURE AND FUNCTION: Translation of Research to Patient Care, 6:1, pp. 18-24, suggested that abnormalities in epidermal barrier functions are linked to acne and a compromised skin barrier can result in further complicating the appearance of acne vulgaris such correlation being important for selecting and determining treatment courses.

Elias et al. describes the application of lipids and lipid formulations for treatment of subjects suffering from skin or mucous membrane diseases or disorders which display epidermal hyperproliferation and disruptions of the barrier function. However, Elias et al. do not apply their teachings specifically to treatment of acne.

### SUMMARY OF THE INVENTION

The present invention relates to implementation of applicants' discovery that certain extracts of Malva neglecta alone or in combination with cholesterol are beneficial for use in topical compositions for application to the skin and in methods of treating skin affected by acne, and provide significant and unexpected benefits for skin, including enhancing skin barrier function, and, inhibiting, reducing, and/or treating appearance and formation of acne on skin (hereinafter referenced as just “reducing at least one sign of acne” for the sake of simplicity, the term “reducing” to be understood to include reducing, inhibiting, treating, delaying, improving, and the like).

Surprisingly, it was discovered that an effective Malva neglecta non-polar extract for improving skin barrier function, and reducing at least one sign of acne on skin uses non-polar solvents. Non-polar solvents may include a solvent selected from the group consisting of liquid carbon dioxide with or without polarity modifier, aqueous ethanol, C₁-C₈ alcohols (such as methanol, ethanol, propanols, and butanols), C₁-C₈ alkanes (such as pentanes, hexanes, and heptanes), C₂-C₈ glycols/polyols (such as glycerine, butylene glycols, and propylene glycols), C₅-C₈ cycloalkanes (such as cyclopentanes, cyclohexanes, and cycloheptanes), C₁-C₈ alkyl ethers, C₁-C₈ aliphatics, ketones, methylene chloride, ethyl acetate, xylene, toluene, vegetable oil, mineral oil, and combinations thereof. In a preferred embodiment, the Malva neglecta is extracted using hexane as a solvent. Applicants have determined that the more non-polar the extract, the more activity and ceramide production induced by the application of Malva neglecta to skin cells.

Accordingly, the present invention is directed to topical compositions comprising non-polar extracts of Malva neglecta for enhancing skin barrier function and reducing at least one sign of acne, and a method of using same for enhancing skin barrier function and reducing at least one sign of acne. In another embodiment, the topical composition includes a non-polar extract of Malva neglecta in combination with cholesterol, and the method includes application of such composition. In yet another embodiment, the topical composition is essentially free of polar components.

Surprisingly, it was also discovered that an effective Malva neglecta extract for improving skin barrier function, and reducing at least one sign of acne on skin uses a lipophilic extract of Malva neglecta.

Accordingly, the present invention is directed to topical compositions comprising lipophilic extracts of Malva neglecta for enhancing skin barrier function and reducing at least one sign of acne. In another embodiment, the topical composition includes lipophilic extracts of Malva neglecta in combination with cholesterol, and the method includes application of such composition.

The topical composition of the present invention preferably includes a cosmetically acceptable topical carrier as well. The topical carrier may include additional active ingredients for treating a skin condition that the Malva neglecta is being used to treat. In particular, any additional active ingredient that may be used to treat acne, or another condition, such ingredient preferably being compatible with the Malva neglecta, may be included in the topical composition of the present invention.

The extract of Malva neglecta to be applied topically to skin to treat a skin condition preferably is a non-polar and/or lipophilic extract of any part of the Malva neglecta plant.

These and other features and advantages of the present invention will be readily apparent from the following detailed description of the invention and accompanying drawings, the scope of the invention being set out in the appended claims.

### BRIEF DESCRIPTION OF THE DRAWINGS

FIG. 1 is a graphical depiction of the amounts of different classes of lipids present in the epidermis of subjects with clear skin and skin affected by acne;

FIG. 2 is a graphical depiction of the amounts of different classes of lipids present in the sebum of subjects with clear skin and skin affected by acne;

FIG. 3 is a graphical depiction of the seasonal changes seen in epidermal lipids and their correlation with acne severity;

FIG. 4 is a graphical depiction of the mean transepidermal water loss in clear skin and acne skin subjects; and

FIG. 5 is a graphical depiction of the skin conductance in clear skin and acne subjects.

### DETAILED DESCRIPTION OF THE INVENTION

It will be appreciated that all percentages listed herein, unless otherwise stated, are weight percentages based on the total weight of the composition.

As used herein, “skin in need of improving skin barrier function” means, without limitation, skin that is lacking in moisture, lacking in or having too much sebum, cracked, dry, itchy, scaly, xerodermic, dehydrated, lacks suppleness, lacks radiance, dull or lacks lipids, has altered free fatty acids:ceramides:cholesterol ratio, has altered transepidermal water loss, has altered water barrier function, has altered skin conductance, epidermal differentiation, increased inflammation/irritation, hyperkeratinization, abnormal desquamation and bacterial proliferation. As used herein, “skin in need of treatment for at least one sign of acne” means, without limitation, skin that is in need of: reduction, inhibition, treatment, delay of the induction of any form of acne; and/or reduction of: redness, irritation, inflammation, hyperkeratonization, bacterial proliferation, and/or abnormal desquamation. Further, as used herein, “improve acne” (and conjugations thereof) is to be understood as reduce, inhibit, treat, and/or delay the formation of any acne-like condition and/or at least one sign of any acne-like condition; and/or to improve the condition of skin affected by acne and/or any acne-like condition.

As used herein, “cosmetically/dermatologically acceptable” means suitable for use in contact with tissues (e.g., the skin or hair) without undue toxicity, incompatibility, instability, irritation, allergic response, and the like.

As used herein, the term “safe and effective amount” means an amount sufficient to induce the desired effect, but low enough to avoid serious side effects. The safe and effective amount of the compound, extract, or composition will vary with, e.g., the age, health and environmental exposure of the end user, the duration and nature of the treatment, the specific extract, ingredient, or composition employed, the particular pharmaceutically-acceptable carrier utilized, and like factors.

As described herein, applicants have discovered that extracts of Malva neglecta and topical compositions containing them provide unexpectedly good skin barrier function, and reduce, inhibit, treat, and delay the formation of any acne-like condition. In particular, applicants have discovered that extracts of Malva neglecta, and, more particularly, certain specific extracts of Malva neglecta, induce production of ceramides by the skin itself, and such induced production of ceramides will have a beneficial effect in reducing at least one sign of acne.

In accordance with principles of the present invention, applicants have focused on balancing ceramides, free fatty acids, and cholesterol levels in the barrier by inducing endogenous synthesis of ceramides to reach the desired balance of ceramides, free fatty acids, and cholesterol that has been associated with a healthy skin barrier, and at least to improve acne. More particularly, application of Malva neglecta, and preferably an extract of Malva neglecta, and most preferably a non-polar extract of Malva neglecta, to skin induces production of ceramides. In skin in need of improving skin barrier function, and particularly in skin in need of treatment for at least one sign of acne, the level of ceramides is either too low, or not present in appropriate proportions relative to free fatty acids and cholesterol. Application of Malva neglecta, and preferably an extract of Malva neglecta, and most preferably a non-polar extract of Malva neglecta results in an increase of ceramides in such skin, which improves skin barrier function and improves acne.

In another embodiment, applicants have discovered that extracts of Malva neglecta and topical compositions containing extracts of Malva neglecta provide unexpectedly good skin barrier function, and improve acne in combination with cholesterol. In particular, skin affected by acne has been found to have elevated levels of free fatty acids, particularly monounsaturated free fatty acids with sebaceous origin, which are believed to be disruptive to the skin barrier. See Mack Correa MC1, Mao G, Saad P, Flach C R, Mendelsohn R, Walters R M, “Molecular interactions of plant oil components with stratum corneum lipids correlate with clinical measures of skin barrier function,” Exp Dermatol. (2014 January) 23(1):39-44. Ceramides, cholesterol, and free fatty acids are the predominant lipids that constitute the skin barrier lipid lamellae. Increasing free fatty acids, particularly monounsaturated ones of sebaceous origin, lead to an imbalance of the overall lipid component of the skin barrier. Applicants have determined that an imbalance of the relative weight percent of ceramides, cholesterol, and free fatty acids detrimentally affects the overall lipid structure and composition of the skin barrier and is correlated with incidence of acne. Applicants have determined that balancing the amounts of ceramides, cholesterol, and free fatty acids in skin affected by acne is as effective a treatment as removing sebum from the affected skin. Accordingly, applicants have found that increasing endogenous ceramide levels by applying extracts of Malva neglecta to skin affected by acne to induce production of ceramides by the affected skin, and adding cholesterol, as described above, is an alternative to removing sebum to treat skin affected by acne and to improve acne.

It will be appreciated that additional ceramide synthesis promoting agents may be used in conjunction with Malva neglecta, and preferably an extract of Malva neglecta, and most preferably a non-polar extract of Malva neglecta in accordance with principles of the present invention to increase the total amount of ceramides on the skin. Examples of suitable ceramide synthesis promoting agents include, but are not limited to, extracts of Bursera simaruba seeds. These ceramide synthesis promoting agents may be present in the composition from about 0.0001 to about 20%, from about 0.001 to about 10%, from about 0.01 to about 5%, from about 0.1 to about 5%, or from about 0.2 to about 2%.

It will further be appreciated that compositions comprising additional ceramides, such as, but not limited to, O-acylceramides, and sphingosine metabolites and derivatives, in conjunction with Malva neglecta, and preferably an extract of Malva neglecta, and most preferably a non-polar extract of Malva neglecta is also contemplated by the present invention for applying to skin in need of treatment of acne.

In accordance with principles of the present invention, compositions comprising certain extracts of Malva neglecta are applied to skin to provide a significant increase in ceramide levels in human skin cells, which is correlated to improved skin barrier function. As noted previously, any disturbances in the ratio of ceramides:free fatty acids:cholesterol are believed to account for a perturbed barrier function. An improved skin barrier function is, therefore, desirable for an overall skin health and specifically for reducing at least one sign of acne. Applicants have discovered that application of extracts of Malva neglecta induce production of ceramides and therefore contribute to improve the skin barrier function and thus improve acne. Moreover, applicants have determined that it is highly desirable to have an appropriate balance of the skin lipids, e.g., ceramides:free fatty acids:cholesterol, to achieve significant improvements in skin barrier structure and function specifically to improve acne. Application of extracts, and, preferably certain extracts of Malva neglecta, and optionally also cholesterol, to skin with acne supports, boosts, fortifies, and otherwise improves (hereinafter simply “improves” for the sake of convenience without intent to limit) skin barrier health to improve acne. Applicants have measured the various skin lipids from the skin of individuals with acne and found that the free fatty acids on the skin of individuals affected by acne were significantly higher than on the skin of individuals with clear skin, as shown in Example 2 herein. The present invention aims at balancing the ratio of skin lipids by providing a combination of Malva neglecta extracts, singly or in combination with cholesterol, to restore the skin barrier to desired relative proportions of ceramides:free fatty acids:cholesterol. Because skin affected by acne typically has high levels of free fatty acids (e.g., ones originating from sebum and others from hydrolysis of sebaceous triglycerides by bacterial lipases), addition of free fatty acids generally is not considered necessary. However, it will be appreciated that the overall balance of ceramides, free fatty acids, and cholesterol has been found by applicants to be important in reducing at least one sign of acne such that addition of free fatty acids may deemed appropriate in certain circumstances.

An altered water barrier function is an indicator of impaired skin barrier function (Yamamoto et al., Arch Dermatol Res., 1995, 287: 214-218). Water barrier function is evaluated by measuring the transepidermal water loss (TEWL) and high frequency skin conductivity test. Applicants have measured the transepidermal water loss (TEWL) and the skin conductance of subjects with acne as described in the Example 5 herein and noted that these values are altered in subjects with acne. In particular, patients with acne typically have increased TEWL and decreased skin conductance as compared with patients without acne. The composition of the present invention is capable of restoring TEWL and skin conductance values, indicative of skin barrier function, to that seen in subjects with clear skin by improving the ceramide levels of the skin. The composition of the present invention this is also capable of restoring the skin barrier function.

Any suitable manner of preparing the extracts of Malva neglecta for use in accordance with the present invention may be used. Suitable extracts may be obtained using conventional methods including, but not limited to, direct extraction from the biomass by grinding, macerating, pressing, squeezing, mashing, centrifuging, and/or processes such as cold percolation, agitation/distillation, microwave assisted extraction, sonication, supercritical/subcritical CO₂ compressed gas extraction with or without polarity modifier, pressurized solvent extraction, accelerated solvent extraction, surfactant assisted pressurized hot water extraction, oil extraction, membrane extraction, Soxhlet extraction, the gold finger distillation/extraction and/or processes disclosed, for example, in U.S. Pat. Nos. 7,442,391, 7,473,435, and 7,537,791 to Integrated Botanical Technologies, LLC, incorporated herein by reference, and the like, or by other methods such as solvent extraction, and the like. In particular, an extract in accordance with the present invention preferably is a solvent-based extraction made by grinding or macerating plant material in a solvent, typically an organic solvent such as an alcohol, acetone, liquid carbon dioxide with or without polarity modifier, hexane, or chloroform. The resulting extract comprised mainly non-polar compounds. The plant biomass preferably is separated entirely from the extraction, and is not used after extraction.

Any of a variety of solvents including aqueous ethanol, liquid carbon dioxide with or without polarity modifier, organic solvents, or combinations of two or more thereof may be used in methods of comprising solvent extraction. Preferably, non-polar organic solvents are used. Suitable non-polar organic solvents are C₁-C₈ alkanes, and, in particular, hexane; C₅-C₈ cycloalkanes; liquid carbon dioxide, C₁-C₈ alcohols, C₂-C₈ glycols/polyols, C₁-C₈ alkyl ethers, in particular, ethyl ether, and petroleum ethers; ketones, including C₃-C₈ ketones, methylene chloride, ethyl acetate, xylene, toluene, chloroform, vegetable oil, mineral oil and the like. Particularly effective, and thus preferred solvents include aqueous ethanol, liquid carbon dioxide, vegetable oil, C₁-C₈ alcohols, C₁-C₈ alkanes, C₂-C₈ glycols/polyols, C₅-C₈ cycloalkanes, and combinations thereof. In certain embodiments, the non-polar extract is extracted from Malva neglecta roots using hexane, glycerine, C₃-C₄ glycols, ethanol, liquid carbon dioxide with or without polarity modifier, chloroform, or a combination thereof. In certain preferred embodiments, the non-polar extract is extracted from Malva neglecta roots using hexanes, ethanol, aqueous ethanol, or liquid carbon dioxide with or without polarity modifier. In certain embodiments, the non-polar extract is extracted from Malva neglecta aerial parts (above-ground parts, e.g., leaves, flowers, shoots, seeds, etc.) using hexane, glycerine, C₃-C₄ glycols, ethanol, aqueous ethanol, liquid carbon dioxide with or without polarity modifier, chloroform, or a combination thereof. In certain preferred embodiments, the non-polar extract is extracted from Malva neglecta aerial parts (above-ground parts, e.g., leaves, flowers, shoots, seeds, etc.) using hexanes, ethanol, aqueous ethanol, or liquid carbon dioxide with or without polarity modifier. In certain embodiments, the non-polar extract is extracted from Malva neglecta whole herb using hexane, glycerine, C₃-C₄ glycols, ethanol, aqueous ethanol, liquid carbon dioxide with or without polarity modifier, chloroform, or a combination thereof. In certain preferred embodiments, the non-polar extract is extracted from Malva neglecta whole herb using hexanes, ethanol, aqueous ethanol, or liquid carbon dioxide with or without polarity modifier. It will be appreciated that non-polar extracts or compounds are not characterized by a dipole, and are extracts that are not ionizing when dissolved in water, a nonionic substance. A non-polar compound can also be defined as a compound comprised of molecules linked through chemical bonds arranged in such a way that the distribution of charges is symmetrical. Non-polar compounds may dissolve in water but would not dissociate into ions, e.g., non-polar amino acids.

In certain preferred embodiments, the extract of the invention is an extract prepared by pulverizing the Malva neglecta raw material and extracting using a solvent having a dielectric constant of a value between about 1 and about 80 at 20° C., preferably a dielectric constant of a value between about 2 and about 60 at 20° C., more preferably a dielectric constant of a value between about 2 and about 40 at 20° C., and even more preferably a dielectric constant of a value between about 2 and 35 at 20° C.

Applicants have further discovered that lipophilic extracts of Malva neglecta and topical compositions containing lipophilic extracts of Malva neglecta provide unexpectedly good skin barrier protection, inhibit, reduce or treat the appearance and formation of acne on skin. More particularly, lipophilic extracts have lipophilic compounds that can serve as PPAR agonists and as such bind to PPAR receptors, which activate a cascade of reactions, with the end result being synthesis of ceramides. Such extracts typically are comprised of lipids from the Malva neglecta plant and are freely soluble and/or extracted with fats, oils, lipids, or solvents such as alkanes, toluene, petroleum ether, or liquid CO₂ with or without polarity modifier. It will be appreciated that lipophilic extracts or compounds are generally not soluble in water and are compounds having an affinity for, tending to combine with, or capable of dissolving in lipids. Lipophilicity, hydrophobicity, and non-polarity can describe the same tendency towards participation in the London dispersion force as the terms are often used interchangeably. However, the terms “lipophilic” and “hydrophobic” are not synonymous, as can be seen with silicones and fluorocarbons, which are hydrophobic but not lipophilic. Moreover, although there is an overlap with lipophilic and non-polar extracts, such extracts can be exclusive as well. For example, non-polar amino acids are not lipophilic in nature, and free fatty acids are lipophilic compounds but are not non-polar. Sterols can be classified as both, e.g., cholesterol. An example of a solvent that results in non-polar but non-lipophilic extracts is ethyl acetate. An example of a solvent that results in lipophilic but not non-polar extracts is hexane.

In certain embodiments, the composition may include extracts from selected parts of Malva neglecta, for example, one or more of the leaves, shoots, roots, fruits, flowers, seeds, or flowers. In other embodiments, the composition may include extracts from the whole herb of Malva neglecta, including leaves, shoots, roots, fruits, flowers, and seeds. Alternatively, the composition may include an extract of the Malva neglecta aerial parts and/or an extract of the Malva neglecta roots.

The present invention further comprises a method of improving the barrier function and improving at least one sign of acne in skin by applying to skin in need of improving skin barrier function and reducing at least one sign of acne an extract of Malva neglecta, in particular an extract of Malva neglecta aerial parts and/or roots. The method comprises for example topically applying a composition of the present invention comprising an extract of Malva neglecta, in particular an extract of Malva neglecta aerial parts and/or roots to skin in need of improving skin barrier function to improve acne. Such topical application may be to any skin in need of treatment on the body, for example skin of the face, lips, neck, chest, back, arms, buttocks, axilla, and/or legs. Preferably, the extract is a non-polar and/or lipophilic extract of Malva neglecta. Even more preferably, the extract is free of polar components. The extract of Malva neglecta is preferably applied in an effective amount that results in inducing production of ceramides to achieve the desired improvement of skin barrier function to result in improvement of acne.

The present invention further comprises a method of improving skin barrier function and improving acne by applying to skin in need of improving skin barrier function and reducing at least one sign of acne an extract of Malva neglecta, in particular an extract of Malva neglecta aerial parts and/or roots, in combination with cholesterol. The method comprises, for example, topically applying a composition of the present invention comprising an extract of Malva neglecta, in particular an extract of Malva neglecta aerial parts and/or roots, in combination with cholesterol to skin in need of improving skin barrier function and reducing at least one sign of acne. Such topical application may be to any skin in need of treatment on the body, for example skin of the face, lips, neck, chest, back, buttocks, arms, axilla, and/or legs. Preferably, the extract is a non-polar and/or lipophilic extract of Malva neglecta. Even more preferably, the extract is free of polar components. The composition is preferably applied in an effective amount that results in the desired increase in production of ceramides to result in improvement of skin barrier function and a reduction of at least one sign of acne.

In accordance with a preferred embodiment. the present invention comprises a method of improving skin barrier function and improving at least one sign of acne in skin by applying to skin in need of improving skin barrier function and reducing at least one sign of acne an extract of Malva neglecta, in particular an extract of Malva neglecta aerial parts and/or roots, in combination with cholesterol. The method comprises, for example, topically applying a composition of the present invention comprising an extract of Malva neglecta, in particular an extract of Malva neglecta aerial parts and/or roots, in combination with cholesterol to skin in need of improving skin barrier function and reducing at least one sign of acne. Such topical application may be to any skin in need of treatment on the body, for example skin of the face, lips, neck, chest, back, buttocks, arms, axilla, and/or legs. Preferably, the extract is a non-polar and/or lipophilic extract of Malva neglecta. The composition is preferably applied in an effective amount that results in the desired increase in production of ceramides to result in improvement of skin barrier function and a reduction of at least one sign of acne.

Any suitable amount of extract of Malva neglecta may be used in the compositions of the present invention. Preferably, the compositions comprise a safe and effective amount of extract of Malva neglecta. In particular, the amount of Malva neglecta extract to be used preferably is selected to achieve the desired treatment of a given skin condition. For instance, the amount of Malva neglecta extract to be used to improve skin barrier function is selected based on the desired effect achieved. Likewise, the amount of Malva neglecta extract to be used to improve at least one sign of acne in skin is selected to achieve the desired amount of improvement. All such amounts are determined by applying the Malva neglecta extract to the skin and observing the effect until the desired results are achieved, thereby determining a therapeutically effective amount of Malva neglecta extract. In one embodiment of the present invention, the amount of extract of Malva neglecta used in a composition of the invention is that effective to achieve an increase in the ceramide levels by at least 1% or higher, preferably about 5% or higher, and more preferably about 10% or higher according to the test Determination of Ceramide Profile by High-Performance Thin-layer Chromatography (Assay 5) described herein. In certain preferred embodiments, the compositions comprise from greater than zero to about 20% extract of Malva neglecta. In certain other preferred embodiments, the compositions comprise from about 0.0001 to about 20%, from about 0.0001 to about 5%, from about 0.001 to about 10%, from about 0.01 to about 5%, from about 0.1 to about 5%, or from about 0.2 to about 2% of extract of Malva neglecta.

The efficacy of the composition comprising Malva neglecta in improving skin barrier function may be determined by confirming an increase in ceramide levels has occurred, and by confirming improved barrier function has been achieved, such as by measuring a decrease in transepidermal water loss (“TEWL”) or increased skin conductance. In one embodiment of the present invention, the amount of extract of Malva neglecta used in a composition of the invention is that effective to achieve an increase in the ceramide levels by at least 1% or higher, preferably about 5% or higher, and more preferably about 10% or higher according to the test Determination of Ceramide Profile by High-Performance Thin-layer Chromatography (Assay 1) described herein.

The efficacy of Malva neglecta in improving skin barrier function and/or improving at least one sign of acne in skin may be measured by assessing transepidermal water loss (TEWL), and skin conductance. In one embodiment of the present invention, the amount of extract of Malva neglecta used in a composition of the invention is that effective for providing a stable decrease of transepidermal water loss (TEWL) by greater than 2-3 g/m²·h, or, more preferably, greater than 5 g/m²·h, over the Baseline/pre-treatment levels, when measured in accordance with the Determination of Transepidermal Water Loss (TEWL) protocol (Assay 2) described herein; and providing any increase in skin conductance in the treated skin over the Baseline/pre-treatment levels, when measured in accordance with the Determination of Skin Conductance (Skicon) procedure (Assay 3) described herein.

In certain preferred embodiments, the compositions comprise from greater than zero to about 20% extract of Malva neglecta. In certain other preferred embodiments, the compositions comprise from about 0.0001 to about 20%, from about 0.001 to about 10%, from about 0.01 to about 5%, from about 0.1 to about 5%, or from about 0.2 to about 2% of extract of Malva neglecta.

Any suitable carrier may be used in the compositions. Preferably, the carrier is a cosmetically-acceptable carrier. As will be recognized by those of skill in the art, cosmetically acceptable carriers comprise carriers that are suitable for use in contact with the body, in particular the skin, without undue toxicity, incompatibility, instability, irritation, allergic response, and the like. A safe and effective amount of carrier is from about 50% to about 99.999%, preferably from about 80% to about 99.9%, more preferably from about 99.9% to about 95%, most preferably from about 98% to about 99.8% of the composition.

The carrier can be in a wide variety of forms. For example, carriers in the form of emulsions, including, but not limited to, oil-in-water, water-in-oil, water-in-oil-in-water, and oil-in-water-in-silicone emulsions, are useful herein. These emulsions can cover a broad range of viscosities, e.g., from about 100 cps to about 200,000 cps.

Examples of suitable cosmetically-acceptable carriers include cosmetically acceptable solvents and materials for cosmetic solutions, suspensions, lotions, creams, serums, essences, gels, toners, sticks, sprays, ointments, liquid washes and soap bars, shampoos, hair conditioners, pastes, foams, mousses, powders, shaving creams, wipes, patches, strips, powered patches, microneedle patches, bandages, hydrogels, film-forming products, facial and skin masks, make-up, liquid drops, and the like. These product types may contain several types of cosmetically-acceptable carriers including, but not limited to solutions, suspensions, emulsions such as microemulsions and nanoemulsions, gels, solids, liposomes, other encapsulation technologies and the like.

The following are non-limitative examples of carriers. Other carriers can be formulated by those of ordinary skill in the art. In one embodiment, the carrier contains water. In a further embodiment, the carrier may also contain one or more aqueous or organic solvents. Examples of organic solvents include, but are not limited to: dimethyl isosorbide; isopropylmyristate; surfactants of cationic, anionic and nonionic nature; vegetable oils; mineral oils; waxes; gums; synthetic and natural gelling agents; alkanols; glycols; and polyols. Examples of glycols include, but are not limited to, glycerin, propylene glycol, butylene glycol, pentalene glycol, hexylene glycol, polyethylene glycol, polypropylene glycol, diethylene glycol, triethylene glycol, capryl glycol, glycerol, butanediol and hexanetriol, and copolymers or mixtures thereof. Examples of alkanols include, but are not limited to, those having from about 2 carbon atoms to about 12 carbon atoms (e.g., from about 2 carbon atoms to about 4 carbon atoms), such as isopropanol and ethanol. Examples of polyols include, but are not limited to, those having from about 2 carbon atoms to about 15 carbon atoms (e.g., from about 2 carbon atoms to about 10 carbon atoms) such as propylene glycol. The organic solvents may be present in the carrier in an amount, based upon the total weight of the carrier, of from about 1 percent to about 99.99 percent (e.g., from about 20 percent to about 50 percent). Water may be present in the carrier (prior to use) in an amount, based upon the total weight of the carrier, of from about 5 percent to about 95 percent (e.g., from about 50 percent to about 90 percent). Solutions may contain any suitable amounts of solvent, including from about 40 to about 99.99%. Certain preferred solutions contain from about 50 to about 99.9%, from about 60 to about 99%, from about 70 to about 99%, from about 80 to about 99%, or from about 90 to 99% of solvent.

A lotion can be made from such a solution. Lotions typically contain at least one emollient in addition to a solvent. Lotions may comprise from about 1% to about 20% (e.g., from about 5% to about 10%) of an emollient(s) and from about 50% to about 90% (e.g., from about 60% to about 80%) of water.

Another type of product that may be formulated from a solution is a cream. A cream typically contains from about 5% to about 50% (e.g., from about 10% to about 20%) of an emollient(s) and from about 45% to about 85% (e.g., from about 50% to about 75%) of water.

Yet another type of product that may be formulated from a solution is an ointment. An ointment may contain a simple base of animal, vegetable, or synthetic oils or semi-solid 10 hydrocarbons. An ointment may contain from about 2% to about 10% of an emollient(s) plus from about 0.1% to about 2% of a thickening agent(s).

The compositions useful in the present invention can also be formulated as emulsions. If the carrier is an emulsion, from about 1% to about 10% (e.g., from about 2% to about 5%) of the carrier contains an emulsifier(s). Emulsifiers may be nonionic, anionic or cationic.

Lotions and creams can be formulated as emulsions. Typically such lotions contain from 0.5% to about 5% of an emulsifier(s), while such creams would typically contain from about 1% to about 20% (e.g., from about 5% to about 10%) of an emollient(s); from about 20% to about 80% (e.g., from 30% to about 70%) of water; and from about 1% to about 10% (e.g., from about 2% to about 5%) of an emulsifier(s).

Single emulsion skin care preparations, such as lotions and creams, of the oil-in-water type, and water-in-oil type are well-known in the art and are useful in the subject invention. Multiphase emulsion compositions, such as the water-in-oil-in-water type or the oil-in-water-in-oil type, are also useful in the subject invention. In general, such single or multiphase emulsions contain water, emollients, and emulsifiers as essential ingredients.

The compositions of this invention can also be formulated as a gel (e.g., an aqueous, alcohol, alcohol/water, or oil gel using a suitable gelling agent(s)). Suitable gelling agents for aqueous and/or alcoholic gels include, but are not limited to, natural gums, acrylic acid and acrylate polymers, and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically contains between about 0.1% and 5%, by weight, of such gelling agents.

The compositions of the present invention can also be formulated into a solid formulation (e.g., a wax-based stick, soap bar composition, powder, or wipe). The composition of the present invention can also be combined with a solid, semi-solid, or dissolvable substrate (e.g., a wipe, mask, pad, glove, or strip).

The compositions of the present invention may further comprise any of a variety of additional cosmetically active agents. Examples of suitable additional active agents include: skin lightening agents, darkening agents, additional anti-aging agents, tropoelastin promoters, collagen promoters, anti-acne agents, shine control agents, anti-microbial agents such as anti-yeast agents, anti-fungal, and anti-bacterial agents, anti-inflammatory agents, anti-parasite agents, external analgesics, sunscreens, photoprotectors, antioxidants, keratolytic agents, detergents/surfactants, moisturizers, nutrients, vitamins, energy enhancers, anti-perspiration agents, astringents, deodorants, hair removers, hair growth enhancing agents, hair growth delaying agents, firming agents, hydration boosters, efficacy boosters, anti-callous agents, agents for skin conditioning, anti-cellulite agents, odor-control agents such as odor masking or pH changing agents, and the like.

Examples of various suitable additional cosmetically acceptable actives include hydroxy acids; benzoyl peroxide; D-panthenol; UV filters such as but not limited to avobenzone (Parsol 1789), bisdisulizole disodium (Neo Heliopan AP), diethylamino hydroxybenzoyl hexyl benzoate (Uvinul A Plus), ecamsule (Mexoryl SX), methyl anthranilate, 4-aminobenzoic acid (PABA), cinoxate, ethylhexyl triazone (Uvinul T 150), homosalate, 4-methylbenzylidene camphor (Parsol 5000), octyl methoxycinnamate (Octinoxate), octyl salicylate (Octisalate), padimate O (Escalol 507), phenylbenzimidazole sulfonic acid (Ensulizole), polysilicone-15 (Parsol SLX), trolamine salicylate, Bemotrizinol (Tinosorb S), benzophenones 1-12, dioxybenzone, drometrizole trisiloxane (Mexoryl XL), iscotrizinol (Uvasorb HEB), octocrylene, oxybenzone (Eusolex 4360), sulisobenzone, bisoctrizole (Tinosorb M), titanium dioxide, zinc oxide; carotenoids; free radical scavengers; spin traps; retinoids and retinoid precursors such as 30 retinol, retinoic acid and retinyl palmitate; ceramides; polyunsaturated fatty acids; essential fatty acids; enzymes; enzyme inhibitors; minerals; hormones such as estrogens; steroids such as hydrocortisone; 2-dimethylaminoethanol; copper salts such as copper chloride; peptides containing copper such as Cu:Gly-His-Lys, coenzyme Q10; amino acids such a proline; vitamins; lactobionic acid; acetyl-coenzyme A; niacin; riboflavin; thiamin; ribose; electron transporters such as NADH and FADH2; and other botanical extracts such as oat, aloe vera, Feverfew, Soy, Shiitake mushroom extracts, and derivatives and mixtures thereof.

In certain preferred embodiments, the skin care compositions comprise an extract of Malva neglecta and at least one additional skin moisturizing active agent.

In certain preferred embodiments, the skin care compositions comprise an extract of Malva neglecta and at least one additional agent for improving at least one sign of acne in skin. Examples of suitable additional agents improving at least one sign of acne in skin include, but are not limited to anti-acne and/or anti-rosacea agents. Examples of anti-acne and anti-rosacea agents include, but are not limited to: retinoids such as tretinoin, isotretinoin, motretinide, adapalene, tazarotene, azelaic acid, and retinol; salicylic acid; benzoyl peroxide; resorcinol; sulfur; sulfacetamide; urea; antibiotics such as tetracycline, clindamycin, metronidazole, and erythromycin; anti-inflammatory agents such as corticosteroids (e.g., hydrocortisone), ibuprofen, naproxen, and hetprofen; and imidazoles such as ketoconazole and elubiol; and salts and prodrugs thereof. Other examples of anti-acne active agents include essential oils, alpha-bisabolol, dipotassium glycyrrhizinate, camphor, β-glucan, allantoin, feverfew, flavonoids such as soy isoflavones, saw palmetto, chelating agents such as EDTA, lipase inhibitors such as silver and copper ions, hydrolyzed vegetable proteins, inorganic ions of chloride, iodide, fluoride, and their nonionic derivatives chlorine, iodine, fluorine, and other valences, synthetic phospholipids and natural phospholipids such as Arlasilk™ phospholipids CDM, SV, EFA, PLN, and GLA (Uniqema, ICI Group of Companies, Wilton, UK), and combinations of two or more thereof.

“Tropoelastin promoters,” as used herein, refers to a class of compounds that possess the biological activity of enhancing the production of tropoelastin. Tropoelastin promoters, according to the present invention, include all natural or synthetic compounds that are capable of enhancing the production of tropoelastin in the human body.

Examples of suitable tropoelastin promoters include, but are not limited to, blackberry extracts, cotinus extracts, feverfew extracts, extracts of Phyllanthus niruri and bimetal complexes having copper and/or zinc constituents. The bimetal complex having copper and/or zinc constituents may be, for example, copper-zinc citrate, copper-zinc oxalate, copperzinc tartarate, copper-zinc malate, copper-zinc succinate, copper-zinc malonate, copper-zinc maleate, copper-zinc aspartate, copper-zinc glutamate, copper-zinc glutarate, copper-zinc fumarate, copper-zinc glucarate, copper-zinc polyacrylic acid, copper-zinc adipate, copper-zinc pimelate, copper-zinc suberate, copper-zinc azealate, copper-zinc sebacate, copper-zinc dodecanoate, or combinations thereof. In a preferred embodiment, the tropoelastin promoter is selected from blackberry extracts, cotinus extracts, feverfew extracts, and combinations thereof. In a particularly preferred embodiment, the tropoelastin promoter is selected from blackberry extracts, feverfew extracts, and combinations thereof.

By “cotinus extract,” it is meant an extract of the leaves of “Cotinus coggygria,” such as a water extract thereof, available from Bilkokoop of Sofia, Bulgaria.

By “blackberry extract,” it is meant a blend of compounds isolated from the plant of the genus Rubus, and preferably Rubus fruticosus. In one embodiment, the compounds are isolated from the flowers of the plant. In a further embodiment, the compounds are isolated from dried flowers of the plant. Such compounds may be isolated from one or more part of the plant (e.g., the whole plant, flower, seed, root, rhizome, stem, fruit and/or leaf of the plant). In a preferred embodiment, the blackberry extract is a blackberry leaf extract. One particularly suitable blackberry extract is produced by extracting the leaves of Rubus fruticosus with a mixture of water and ethanol compounded to an activity of about 5% to about 10%, with a maltodextrin matrix, commercially available from Symrise Inc. of Teterboro, N.J., and is sold under the name “SymMatrix.”

Extracts of “Phyllanthus niruri” may be harvested and used as the whole plant, or optionally one or more parts of the plant (e.g., flower, seed, root, rhizome, stem, fruit and/or leaf of the plant) may be used. The Phyllanthus niruri plant or parts thereof may be finely divided, such as by grinding or milling, to a powder. A suitable milled form of Phyllanthus niruri is commercially available from Raintree Nutrition, Inc., of Carson City, Nev. Preferably, a low molecular weight fraction of Phyllanthus niruri is used, for instance a fraction of Phyllanthus niruri substantially free of molecular species having a molecular weight of greater than about 100,000 daltons. Preferably, such low molecular weight fraction is water extractable from the Phyllanthus niruri plant.

Compositions of the present invention may include a cosmetically effective amount of one or more tropoelastin promoters such as those described above. The compositions preferably include, on an active basis, from about 0.1% to about 10% of the tropoelastin promoters, more preferably from about 0.5% to about 5% of tropoelastin promoters, and most preferably from about 0.5% to about 2% of the tropoelastin promoters.

“Collagen promoter,” as used herein, refers to compounds that possess the biological activity of enhancing the production of collagen. “Non-retinoid collagen promoters” according to the present invention include all natural or synthetic compounds that are not retinoids, or derived from retinoids, and are capable of enhancing the production of collagen in the human body.

Examples of suitable collagen promoters include, but are not limited to the following: Retinoids including retinol, retinaldehyde, and retinoic acid, extracts of feverfew (Tanacetum parthenium), extracts of Centella asiatica, and extracts of Siegesbeckia orientalis; extracts of soy; collagen-promoting peptides; ursolic acid; and asiaticoside.

Centella asiatica, also known as Violette marronne on Reunion Island, Gotu Kola or Indian pennywort in India, Centella repanda in North America, and Talapetraka in Madagascar, is a polymorphous herb and belongs to the family of Umbelliferae (Apiaceae), particularly to the Hydrocotyle subfamily. It grows wild throughout the tropics and prefers moist and shady regions at an altitude of about 600 to 1200 meters above sea level. Centella asiatica has three varieties: Typica, Abyssinica, and Floridana. The herb is known and used for its healing, sedative, analgesic, antidepressant, antiviral and antimicrobial properties. The biological activity of the herb appears to be due to the presence of triterpene molecules in the herb. A suitable extract of Centella asiatica is available as TECA from Bayer Consumer HealthCare of Basel, Switzerland.

By “extracts of Siegesbeckia orientalis,” is meant any of various extracts of the plant Siegesbeckia orientalis, including Darutoside available from Sederma (Croda International Group of Edison, N.J.).

Suitable collagen-promoting peptides include the following matrikine peptides, (i.e., a peptide derived from the degradation of extracellular matrix proteins—collagen, elastin, or proteoglycan) including palmitoyl pentapeptides, in particular Pal-Lys-Thr-Thr-Lys-Ser-OH, available as MATRIXYL from Sederma (Croda International Group of Edison, N.J.); GHK copper peptide available as PROCYTE from Photomedex of Montgomeryville, Pa.; Palmitoyl GHK peptide available as Biopoeptide CL from Sederma (Croda International Group of Edison, N.J.); Biomimetic tetrapeptides, such as those available as Chronoline Tri Peptide from Unipex of Québec, Canada; and Palmitoyl tri-peptide, available as Syn-Coll from DSM of Basel, Switzerland.

Ursolic acid is also known as pentacyclic triterpene acid, Prunol, Malol, Urson, betaursolic acid and 3-Beta-Hydroxy-Urs-12-En-28-Oic Acid. It is commercially available for example from Sigma-Aldrich of St. Louis, Mo.

Asiaticoside, also known chemically as: [6-[[3,4-dihydroxy-6-(hydroxymethyl)-5-(3,4,5-trihydroxy-6-methyloxan-2-yl)oxyoxan-2-yl]oxymethyl]-3,4,5-trihydroxyoxan-2-yl]10,11-dihydroxy-9-(hydroxymethyl)-1,2,6a,6b,9,12a-hexamethyl-2,3,4,5,6,6a,7,8,8a,10,11,12,13,14b-tetradecahydro-1H-picene-4-a-carboxylate) is commercially available for example from Bayer Santé Familiale Division Serdex, 69, Boulevard Victor Hugo 93400 SAINT-OUEN France.

Compositions of the present invention may include a cosmetically effective amount of one or more collagen promoters. The compositions preferably include, on an active basis, from about 0.1% to about 10% of the collagen promoters, more preferably from about 0.5% to about 5% of collagen promoters, and most preferably from about 0.5% to about 2% of the collagen promoters.

The compositions of the present invention may further comprise at least one skin lightening active agent. Examples of suitable skin lightening active agents include, but are not limited to, tyrosinase inhibitors, melanin-degradation agents, melanosome transfer inhibiting agents including PAR-2 antagonists, exfoliants, sunscreens, retinoids, antioxidants, Tranexamic acid, tranexamic acid cetyl ester hydrochloride, skin bleaching agents, linoleic acid, adenosine monophosphate disodium salt, Chamomilla extract, allantoin, opacifiers, talcs and silicas, zinc salts, and the like, and other agents as described in Solano et al. Pigment Cell Res. 19 (550-571) and Ando et al. Int J Mol Sci 11 (2566-2575).

Examples of suitable tyrosinase inhibitors include but, are not limited to, Vitamin C and its derivatives, Vitamin E and its derivatives, Kojic Acid, Arbutin, resorcinols, hydroquinone, Flavones e.g. Licorice flavanoids, Licorice root extract, Mulberry root extract, Dioscorea Coposita root extract, Saxifraga extract and the like, Ellagic acid, Salicylates and derivatives, Glucosamine and derivatives, Fullerene, Hinokitiol, Dioic acid, Acetyl glucosamine, 5,5′-dipropyl-biphenyl-2,2′-diol (Magnolignan), 4-(4-hydroxyphenyl)-2-butanol (4-HPB), combinations of two or more thereof, and the like. Examples of vitamin C derivatives include, but are not limited to, ascorbic acid and salts, Ascorbic Acid-2-Glucoside, sodium ascorbyl phosphate, magnesium ascorbyl phosphate, and natural extract enriched in vitamin C Examples of vitamin E derivatives include, but are not limited to, alpha-tocopherol, beta, tocopherol, gamma-tocopherol, delta-tocopherol, alpha-tocotrienol, beta-tocotrienol, gamma-tocotrienol, delta-tocotrienol and mixtures thereof, tocopherol acetate, tocopherol phosphate and natural extracts enriched in vitamin E derivatives. Examples of resorcinol derivatives include, but are not limited to, resorcinol, 4-substituted resorcinols like 4-alkylresorcinols such as 4-butyresorcinol (rucinol), 4-hexylresorcinol (Synovea HR, Sytheon), phenylethyl resorcinol (Symwhite, Symrise), 1-(2,4-dihydroxyphenyl)-3-(2,4-dimethoxy-3-methylphenyl)-Propane (nivitol, Unigen) and the like and natural extracts enriched in resorcinols. Examples of salicylates include, but are not limited to, 4-methoxy potassium salicylate, salicylic acid, acetylsalicylic acid, 4-methoxysalicylic acid and their salts. In certain preferred embodiments, the tyrosinase inhibitors include a 4-substituted resorcinol, a vitamin C derivative, or a vitamin E derivative. In more preferred embodiments, the tyrosinase inhibitor comprises Phenylethyl resorcinol, 4-hexyl resorcinol, or ascorbyl-2-glucoside.

Examples of suitable melanin-degradation agents include, but are not limited to, peroxides and enzymes such as peroxidases and ligninases. In certain preferred embodiments, the melanin-inhibiting agents include a peroxide or a ligninase.

Examples of suitable melanosome transfer inhibiting agents including PAR-2 antagonists such as soy trypsin inhibitor or Bowman-Birk Inhibitor, Vitamin B3 and derivatives such as Niacinamide, Essential soy, Whole Soy, Soy extract. In certain preferred embodiments, the melanosome transfer inhibiting agents includes a soy extract or niacinamide.

Examples of exfoliants include, but are not limited to, alpha-hydroxy acids such as lactic acid, glycolic acid, malic acid, tartaric acid, citric acid, or any combination of any of the foregoing, beta-hydroxy acids such as salicylic acid, polyhydroxy acids such as lactobionic acid and gluconic acid, and mechanical exfoliation such as microdermabrasion. In certain preferred embodiments, the exfoliants include glycolic acid or salicylic acid.

Examples of sunscreens include, but are not limited to, avobenzone (Parsol 1789), bisdisulizole disodium (Neo Heliopan AP), diethylamino hydroxybenzoyl hexyl benzoate (Uvinul A Plus), ecamsule (Mexoryl SX), methyl anthranilate, 4-aminobenzoic acid (PABA), cinoxate, ethylhexyl triazone (Uvinul T 150), homosalate, 4-methylbenzylidene camphor (Parsol 5000), octyl methoxycinnamate (Octinoxate), octyl salicylate (Octisalate), padimate O (Escalol 507), phenylbenzimidazole sulfonic acid (Ensulizole), polysilicone-15 (Parsol SLX), trolamine salicylate, Bemotrizinol (Tinosorb S), benzophenones 1-12, dioxybenzone, drometrizole trisiloxane (Mexoryl XL), iscotrizinol (Uvasorb HEB), octocrylene, oxybenzone (Eusolex 4360), sulisobenzone, bisoctrizole (Tinosorb M), titanium dioxide, zinc oxide, and the like.

Examples of retinoids include, but are not limited to, retinol (Vitamin A alcohol), retinal (Vitamin A aldehyde), retinyl acetate, retinyl propionate, retinyl linoleate, retinoic acid, retinyl palmitate, isotretinoin, tazarotene, bexarotene, Adapalene, combinations of two or more thereof and the like. In certain preferred embodiments, the retinoid is selected from the group consisting of retinol, retinal, retinyl acetate, retinyl propionate, retinyl linoleate, and combinations of two or more thereof. In certain more preferred embodiments, the retinoid is retinol.

Examples of antioxidants include, but are not limited to, water-soluble antioxidants such as sulfhydryl compounds and their derivatives (e.g., sodium metabisulfite and N-acetyl cysteine, glutathione), lipoic acid and dihydrolipoic acid, stilbenoids such as resveratrol and derivatives, lactoferrin, iron and copper chelators and ascorbic acid and ascorbic acid derivatives (e.g., ascobyl-2-glucoside, ascorbyl palmitate and ascorbyl polypeptide). Oil-soluble antioxidants suitable for use in the compositions of this invention include, but are not limited to, butylated hydroxytoluene, retinoids (e.g., retinol and retinyl palmitate), tocopherols (e.g., tocopherol acetate), tocotrienols, and ubiquinones. Natural extracts containing antioxidants suitable for use in the compositions of this invention, include, but not limited to, extracts containing flavonoids and isoflavonoids and their derivatives (e.g., genistein and diadzein), extracts containing resveratrol and the like. Examples of such natural extracts include grape seed, green tea, black tea, white tea, pine bark, feverfew, parthenolide-free feverfew, oat extracts, blackberry extract, cotinus extract, soy extract, pomelo extract, wheat germ extract, Hesperedin, Grape extract, Portulaca extract, Licochalcone, chalcone, 2,2′-dihydroxy chalcone, Primula extract, propolis, and the like.

The additional cosmetically active agent may be present in a composition in any suitable amount, for example, in an amount of from about 0.0001% to about 20% by weight of the composition, e.g., about 0.001% to about 10% such as about 0.01% to about 5%. In certain preferred embodiments, in an amount of 0.1% to 5% and in other preferred embodiments from 1% to 2%.

Compositions of the present invention may include a cosmetically effective amount of one or more anti-inflammatory compounds.

Examples of suitable anti-inflammatory agents include substituted resorcinols, (E)-3-(4-methylphenylsulfonyl)-2-propenenitrile (such as “Bay 11-7082,” commercially available from Sigma-Aldrich of St. Louis, Mo.), tetrahydrocurcuminoids (such as Tetrahydrocurcuminoid CG, available from Sabinsa Corporation of Piscataway, N.J.), extracts and materials derived from the following: Phellodendron amurense Cortex Extract (PCE), Non-Denatured Soy (Glycine max), Feverfew (Tanacetum parthenium), Ginger (Zingiber officinale), Ginko (Ginkgo biloba), Madecassoside (Centella asiatica extract ingredient), Cotinus (Cotinus coggygria), Butterbur Extract (Petasites hybridus), Goji Berry (Lycium barbarum), Milk Thistle Extract (Silybum marianum), Honeysuckle (Lonicera japonica), Basalm of Peru (Myroxylon pereirae), Sage (Salvia officinalis), Cranberry Extract (Vaccinium oxycoccos), Amaranth Oil (Amaranthus cruentus), Pomegranate (Punica granatum), Yerbe Mate (Ilex paraguariensis Leaf Extract), White Lily Flower Extract (Lilium candidum), Olive Leaf Extract (Olea europaea), Phloretin (apple extract), Oat Flour (Aveena sativa), Lifenol (Hops: Humulus lupulus) Extract, Bugrane P (Ononis spinosa), Licochalcone (Licorice: Glycyrrhiza inflate extract ingredient), Symrelief (Bisabolol and Ginger extract), combinations of two or more thereof, and the like.

In one embodiment, the anti-inflammatory agent is a resorcinol. Particularly suitable substituted resorcinols include 4-hexyl resorcinol and 4-octylresorcinol, particularly 4-hexyl resorcinol. 4-Hexyl resorcinol is commercially available as “SYNOVEA HR” from Sytheon of Lincoln Park, N.J. 4-Octylresorcinol is commercially available from City Chemical LLC of West Haven, Conn.

By “extracts of feverfew,” it is meant extracts of the plant “Tanacetum parthenium,” such as may be produced according to the details set for the in US Patent Application Publication No. 2007/0196523, entitled “PARTHENOLIDE FREE BIOACTIVE INGREDIENTS FROM FEVERFEW (TANACETUM PARTHENIUM) AND PROCESSES FOR THEIR PRODUCTION.” One particularly suitable feverfew extract is commercially available as about 20% active feverfew, from Integrated Botanical Technologies of Ossining, N.Y.

In the skin care composition of the invention, the ratio of the amounts of the extract of Malva neglecta to the anti-inflammatory compound may be varied. For example, the extract and the anti-inflammatory compound may be present in a weight ratio (which is determined by dividing the amount by weight of the dry extract by the amount by weight of the anti-inflammatory compound) of about 0.001 to about 100, preferably about 0.01 to about 10, more preferably about 0.25 to about 2.

A variety of other materials may also be present in the compositions of the present invention. In certain preferred embodiments, the composition comprises one or more topical ingredients selected from the group consisting of: surfactants, chelating agents, emollients, humectants, conditioners, preservatives, opacifiers, fragrances and the like.

What is meant by an emollient is a compound that helps to maintain the soft, smooth, and pliable appearance of the skin (e.g., by remaining on the skin surface or in the stratum corneum to act as a lubricant). Examples of suitable emollients include those found in Chapter 35, pages 399-415 (Skin Feel Agents, by G Zocchi) in Handbook of Cosmetic Science and Technology (edited by A. Barel, M. Paye and H. Maibach, Published in 2001 by Marcel Dekker, Inc New York, N.Y.), and include, but are not limited to, petrolatum, hexyldecyl stearate and plant, nut, and vegetable oils such as macadamia nut oil, rice bran oil, grape seed oil, palm oil, prim rose oil, hydrogenates peanut oil, and avocado oil.

What is meant by a humectant is a compound intended to increase the water content of the top layers of skin (e.g., hygroscopic compounds). Examples of suitable humectants include those found Chapter 35, pages 399-415 (Skin Feel Agents, by G Zocchi) in Handbook of Cosmetic Science and Technology (edited by A. Barel, M. Paye and H. Maibach, Published in 2001 by Marcel Dekker, Inc New York, N.Y.) and include, but are not limited to, glycerin, sorbitol or trehalose (e.g., α,α-trehalose, β,β-trehalose, α,β-trehalose) or a salt or ester thereof (e.g., trehalose 6-phosphate).

What is meant by a surfactant is a surface-active agent intended to cleanse or emulsify. Examples of suitable surfactants include those found in Chapter 37, pages 431-450 (Classification of surfactants, by L. Oldenhove de Guertechin) in Handbook of Cosmetic Science and Technology (edited by A. Barel, M. Paye and H. Maibach, Published in 2001 by Marcel Dekker, Inc., New York, N.Y.) and include, but are not limited to anionic surfactants such as sulfates, cationic surfactants such as betaines, amphoteric surfactants such as sodium coco glycinate, noionic surfactants such as alkyl polyglucosides.

Examples of suitable chelating agents include those which are capable of protecting and preserving the compositions of this invention. Preferably, the chelating agent is ethylenediamine tetracetic acid (“EDTA”), and more preferably is tetrasodium EDTA, available commercially from Dow Chemical Company of Midland, Mich. under the trade name, “Versene 100XL.”

Suitable preservatives include, for example, parabens, quaternary ammonium species, phenoxyethanol, benzoates, DMDM hydantoin, organic acids and are present in the composition in an amount, based upon the total weight of the composition, from about 0 to about 1 percent or from about 0.05 percent to about 0.5 percent.

Any of a variety of conditioners which impart additional attributes, such as gloss to the hair, are suitable for use in this invention. Examples include, but are not limited to, volatile silicone conditioning agent having an atmospheric pressure boiling point less than about 220° C. Examples of suitable volatile silicones nonexclusively include polydimethylsiloxane, polydimethylcyclosiloxane, hexamethyldisiloxane, cyclomethicone fluids such as polydimethylcyclosiloxane available commercially from Dow Corning Corporation of Midland, Mich. under the tradename, “DC-345” and mixtures thereof, and preferably include cyclomethicone fluids. Other suitable conditioners include cationic polymers, including polyquarterniums, cationic guar, and the like.

Any of a variety of commercially available pearlescent or opacifying agents are suitable for use in the composition. Examples of suitable pearlescent or opacifying agents include, but are not limited to, mono or diesters of (a) fatty acids having from about 16 to about 22 carbon atoms and (b) either ethylene or propylene glycol; mono or diesters of (a) fatty acids having from about 16 to about 22 carbon atoms (b) a polyalkylene glycol of the formula: HO-(JO)ₐ—H, wherein J is an alkylene group having from about 2 to about 3 carbon atoms; and a is 2 or 3; fatty alcohols containing from about 16 to about 22 carbon atoms; fatty esters of the formula: KCOOCH₂L, wherein K and L independently contain from about 15 to about 21 carbon atoms; inorganic solids insoluble in the shampoo composition, and mixtures thereof.

Any fragrance compositions suitable for use on skin may be used in accord with the present invention.

In certain preferred embodiments, the present invention is in the form of a substrate comprising a composition of the present invention. Any suitable substrate may be used. Examples of suitable substrates and substrate materials are disclosed, for example, in U.S. Published Application Nos. 2005/0226834 and 2009/0241242 which are incorporated herein by reference in their entirety.

In certain preferred embodiments, the substrate is a wipe, glove, or a facial mask. Preferably, such embodiments comprise a water-insoluble substrate as such is defined in the cited references above. For certain embodiments, the water-insoluble substrate may have a size and shape such that it covers the face of a human user to facilitate placing the water-insoluble substrate about the face of the user as a mask substrate. For example, the water-insoluble mask substrate may have openings for a mouth, nose, and/or eyes of the user. Alternatively, the water insoluble substrate may have no such openings. Such a configuration without openings may be useful for embodiments of the invention in which the water-insoluble substrate is intended to be draped over a non-facial expanse of skin or if the water-insoluble substrate is intended to be used as wipe. The water-insoluble substrate may have various shapes, such as an angular shape (e.g., rectangular) or an arcuate shape such as circular or oval. For certain embodiments, the substrate is a glove such as described in U.S. Published Application No 2006/0141014 which is incorporated herein in its entirety. In one embodiment of the invention, the product includes a plurality of water-insoluble substrates of different shapes.

Any suitable method of applying the composition to the skin in need may be used. For example, the composition may be applied directly from a package to the skin in need, by hand to the skin in need, or may be transferred from a substrate such as a wipe or mask, or a combination of two or more thereof. In other embodiments, the composition may be applied via a dropper, tube, roller, spray, and patch or added to a bath or otherwise to water to be applied to the skin, and the like. The composition may be applied in a variety of manners/forms, including, without limitation, as a leave-on cream, mask, and/or serum.

In certain preferred embodiments, the methods of the present invention comprise applying at least two different compositions or products comprising a Malva neglecta extract singly or in combination with cholesterol to the skin. For example, the methods may comprise applying a first composition comprising Malva neglecta extract singly or in combination with cholesterol to skin in need of improving skin barrier efficacy and reducing at least one sign of acne, followed by applying a second composition comprising Malva neglecta extract, singly or in combination with cholesterol that is different from the first composition, to the skin in need of treatment. In certain preferred embodiments, the first and second composition may be independently selected from the group consisting of lotions, cleansers, masks, wipes, creams, serums, gels, and the like. In certain preferred embodiments, at least one of the first and second compositions is a cleanser, lotion, cream, essence, or serum, and the other is a facial mask or wipe. In certain other preferred embodiments, at least one of the first and second compositions is a cleanser and the other is a lotion or cream.

While the foregoing description and drawings represent exemplary embodiments of the present invention, it will be understood that various additions, modifications and substitutions may be made therein without departing from the spirit and scope of the present invention. In particular, it will be clear to those skilled in the art that the present invention may be embodied in other specific forms, structures, arrangements, proportions, and with other elements, materials, and components, without departing from the spirit or essential characteristics thereof. One skilled in the art will appreciate that the invention may be used with many modifications of structure, arrangement, proportions, materials, and components and otherwise, used in the practice of the invention, which are particularly adapted to specific environments and operative requirements without departing from the principles of the present invention. The presently disclosed embodiments are therefore to be considered in all respects as illustrative and not restrictive, the scope of the invention being indicated by the appended claims, and not limited to the foregoing description. It will be appreciated that in the claims, the term “comprises/comprising” does not exclude the presence of other elements or steps. In addition, singular references do not exclude a plurality. The terms “a”, “an”, “first”, “second”, etc., do not preclude a plurality.

### EXAMPLES

The following test methods were used in the Examples.

### Assay 1: Determination of Ceramide Profile by High-Performance Thin-Layer Chromatography

### Sample Extraction and Condensation

Skin equivalents or 0.5-1×10⁶ cells were homogenized with 2 mL chloroform:methanol (2:1) and transferred to a vial containing 1 mL Phosphate-Buffered Saline Solution. Homogenizer was rinsed with two consecutive 2 mL portions of chloroform:methanol (2:1) and the rinses were added to the vial containing the extract and the PBS. The mixture was vortexed and the phases were allowed to separate. The organic phase was evaporated to dryness under vacuum. Sample residue dissolved in 200 μL chloroform:methanol (2:1).

### High-Performance Thin-Layer Chromatography

The residue was dissolved in 2004, chloroform:methanol (2:1). Twenty (20) microliters and forty (40) μL of sample solution was applied on the HPTLC plate (Whatman Partisil) using CAMAG Automatic TLC Sampler 4 and separated using the following sequential development system: (1) dichloromethane:ethyl acetate:acetone (80:16:4), (2) chloroform:methanol:acetone (76:16:8), and (3) hexane:chloroform:acetic acid:acetone:methanol (6:80:0.1:10:4). The plates were stained with 3% copper acetate in 8% phosphoric acid and charred at 160° C.

### Quantification

Samples were applied in parallel for positional corrections and compared to a similarly prepared blank extract (tape strip without exposure to skin lipids). Quantification was performed against known quantities of Ceramide III standard (Cosmoferm) by densitometry (CAMAG).

### Assay 2: Determination of Transepidermal Water Loss (TEWL)

An open-chambered evaporimeter was used to measure transepidermal water loss (TEWL), which provided an estimation of skin barrier function in clear skin (control) and acne skin subjects. For these measurements, a probe was placed in gentle contact with the skin surface. The subjects were asked to recline during the evaluation and the probe was kept level; each measurement took about 1 minute. Measurements on the forehead were taken prior to the forehead being wiped for Sebutape® application (SEBUTAPE® is a registered trademark of CUDERM CORPORATION, 12221 Merit Dr., Suite 940, Dallas, Tex. 75251 USA).

### Assay 3: Determination of Skin Conductance (Skicon)

A Skicon 200EX instrument (manufactured by IBS Ltd., of Hamamatsu, Japan) was used to measure the conductance of the skin surface, which helped to provide a measure of skin water content in clear skin (control) and acne skin subjects. For these measurements, a probe was placed in gentle contact with the skin surface. Measurements on the forehead were taken prior to the forehead being wiped for Sebutape® application.

### Assay 4: Determination of Epidermal Lipids

Leukoflex tapes were used to tape strip the skin for analysis of epidermal lipids via HPTLC High Performance Thin Layer Chromatography in subjects with clear skin (control group) and acne skin (test group). The tape-stripping protocol was done as follows:
1. Gloves were worn by study personnel at all times when handling the Leukoflex tapes to prevent possible contamination of the tapes. Unused tapes were kept on a flat surface within a protective case to prevent tapes from coming off of their backing.
2. Tapes were pre-cut in circular shapes (˜3.5 cm diameter). Using forceps, the tape was removed from the backing and applied to test area as tautly as possible.
3. Even contact of the tape was ensured by rolling a small wooden roller or one gloved finger (once) across the tape.
4. Using forceps, the tape was removed 60 seconds after application.
5. The tape was insert into 20 mL glass scintillation vials (to avoid folding the tape in on itself as best as possible).
6. Samples were stored at −80° C. until analysis or shipment.

### Assay 5: Determination of Sebaceous Lipids

Sebutape® Adhesive Patches were used to assess sebum recovery (i.e. the amount of sebum produced in a given period), according to the following procedure:
1. Subjects were acclimated to room conditions [Proof-of-Principle (POP) lab] for about 30 minutes, with each visit made at approximately the same time of day to avoid Circadian effects.
2. Test area (forehead) was wiped with an alcohol wipe (BD 70% IPA—i.e., a Becton Dickinson IsoPropanol wipe) ten times to remove any sebum/debris/sweat/product, etc. The same technique was used for each subject/time point. In a horizontal fashion, the area was wiped 5 times from left to right, the wipe was flipped over, and then the area was wiped 5 times from right to left, being sure to cover the entire sampling area.
3. Test area was allowed to dry for at least one minute.
4. Gloves were worn by study personnel at all times to prevent possible contamination of the Sebutapes by oil from the fingertips. Using flat-edged tweezers, 2 CuDerm Sebutape Adhesive Patches were firmly applied to the test area in a vertical orientation, with one tape placed over either eyebrow near center of face. The tapes were pressed firmly into place by rolling a gloved finger over each tape.
5. Timer is set for 30 minutes, during which the subject was required to stay in the POP room or adjoining instrumentation room (which was set for the same temperature/humidity).
6. After the 30 minute oil-recovery period, the tapes were removed using tweezers and applied to a Sebutape card for image analysis.
7. A second set of tapes was applied as in Step 4 in the same position as the original tapes (an outline remains from first set).
8. Timer is set for 30 minutes, during which the subject is required to stay in the POP room or adjoining instrumentation room (which is set for the same temperature/humidity).
9. After 30 minutes, the tapes are removed using tweezers.
10. Tapes from the second set are placed directly into 20 mL scintillation vials and kept on ice until they can be stored in a −80° C. freezer for later lipid analysis of separation by HPTLC and further quantification by GC-FID (Gas Chromatography—Flame Ionization Detection).
   Note: all tapes (on cards and in vials) are labeled according to subject number, time point, and position on subject's forehead (see Attachment III, with left/right defined as subject's left/right side).

### Assay 6: Gene Expression

Samples were isolated from primary human keratinocytes and skin equivalents that had been treated with extracts dissolved in DMSO (Dimethyl sulfoxide) or DMSO without extracts (as control) for 24 hours using Qiagen RNeasy kit with DNase I digestion (Cat#79254) (Valencia, Calif.). Reverse transcription was performed using High Capacity cDNA kit (Life technologies Cat#4368814).

40 to 60 ng of cDNA samples were used for QPCR (quantitative polymerase chain reaction). Taqman gene expression assay was purchased from Life Technologies (Grand Island, N.Y.). QPCR reaction was performed using ABI 7500 fast amplifier manufactured by Applied Biosystems®. The PCR primers used are presented in Table 1. All gene expression data were normalized by reference genes, polymerase (RNA) II polypeptide A (POLR2A) or/and ribosomal protein, large, PO(RPLPO). Relative gene expression was calculated by comparative CT method.

Sphingomyelin phosphodiesterase 3 is an enzyme that in humans is encoded by the SMPD3 gene and is involved in ceramide synthesis. Ceramide glucosyltransferase (UGCG) converts ceramides to glucosylceramides for transport. Elongation of very long chain fatty acids 4 (ELOVL4) is required for very long chain fatty acids synthesis, which are a major component of ceramides.

TABLE 1
PCR primers
Life Technologies (Applied biosystems)

| Gene Symbol | Catalog Number |
|---|---|
| SMPD3 | Hs00920354_m1 |
| GBA | Hs00986836_g1 |
| SPTLC2 | Hs00191585_m1 |
| ABCA12 | Hs00917552_m1 |
| ELOVL4 | Hs00224122_m1 |
| UGCG | Hs00234293_m1 |
| CERS3 | Hs00698859_m1 |

The following examples illustrate the preparation and efficacy of Malva neglecta extracts.

### Example 1

### Preparation of Malva neglecta Extract from Aerial Parts (E1)

Malva neglecta plants were wild-collected in New York. Species identification was based on gross morphological characteristics [Gleason & Cronquist, Manual of Vascular Plants; D Van Nostrand Company, NY: p. 462-463]. Plants were cleaned of soil and debris and separated into aerial parts and roots. Approximately 80 g of fresh aerial plant material was homogenized in a blender with 200 mL of 80% aqueous methanol; the suspension was maintained in constant motion for 24 hours. The resulting suspension was then filtered and dried under low pressure using a rotary evaporator not exceeding 40° C. After filtration, the left over raw material was again extracted as described above. The combined dry mass from both extractions was designated the crude extract, approximately 2.1 g, for a yield of 6.6%. The crude extract was resuspended in 100 mL water and subjected to liquid-liquid solvent partitioning in a separatory funnel using three equal parts hexane. The three hexane partitions were combined and dried under low pressure using a rotary evaporator not exceeding 40° C. to achieve a total mass of approximately 200 mg (E1), for a yield of 0.6%.

### Example 2

### Estimation of Epidermal and Sebum Lipids

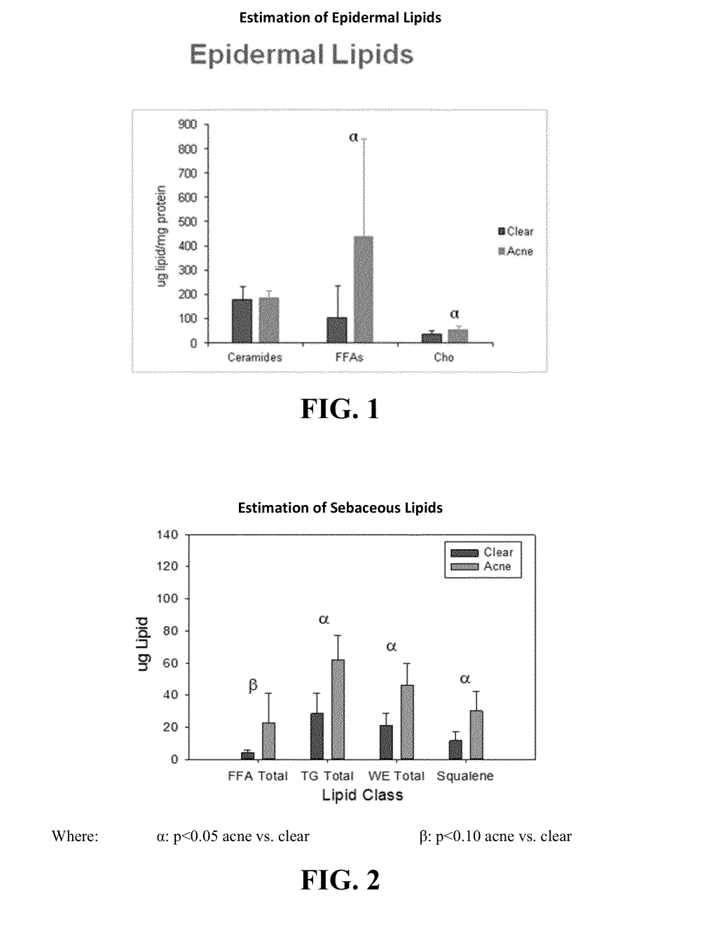

The epidermal lipids and sebum lipids were measured using the method of Assay 4 and Assay 5 respectively in subjects with acne skin and the results are plotted in graphs 1 and 2 depicted in FIGS. 1 and 2, respectively.

As seen from the graphs_depicted in FIGS. 1 and 2, the Free Fatty Acid class is much higher in the acne group than the control clear skin group in the epidermal or surface lipids. The Free Fatty Acids in the sebum are also higher in acne subjects than the clear skin subjects. All sebaceous lipid classes are proportionally higher in acne subjects.

### Example 3

### Determination of Ceramides in Human Primary Keratinocytes

Extract E1 was tested for ceramide levels using the method of Assay 1 described above. The results are given in Table 2 below.

TABLE 2
Results of ceramide production in a keratinocyte cell culture model

| Extract | Concentration (μg/mL) | Percent of control |
|---|---|---|
| Vehicle | 0.1% DMSO | 100 |
| E1 | 25 | 124 |

### Example 4

### Transcription of Ceramide Synthesis Genes

Extracts E1 was tested for increase in ceramide synthesis gene transcription, in accord with the method of Assay 6 described above and the results are given in Table 3 below.

TABLE 3
Results of PCR experiments using human keratinocyte cell culture showing
results for ceramide synthesis and transport genes.

| | | SMPD3 | GBA | SPTLC2 | ABCA12 | ELOVL4 | UGCG | CERS3 |
|---|---|---|---|---|---|---|---|---|
| Test | Concentration | Fold | Fold | Fold | Fold | Fold | Fold | Fold |
| Article | (μg/mL) | Change | Change | Change | Change | Change | Change | Change |
| Control | VEH (0.5% | 1.0 | 1.0 | 1.0 | 1.0 | 1.0 | 1.0 | 1.0 |
| | DMSO) | | | | | | | |
| E1 | 25 | 9.1 | 3.1 | 1.3 | 4.2 | 5.7 | 7.0 | 3.9 |

### Example 5

### Correlation of Epidermal Lipid Levels with Acne Severity

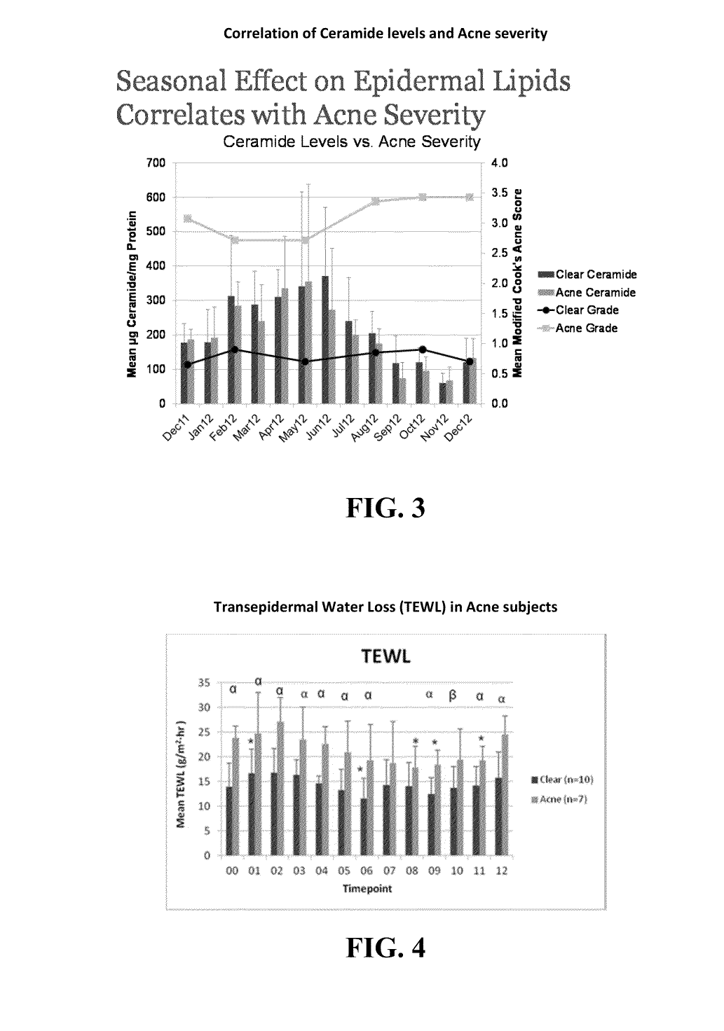

The graph depicted in FIG. 3—shows that high ceramides correlate with lower acne score and vice versa. Acne severity score was determined by a dermatologist. Monthly variations reflect seasonal changes affecting the skin.

### Example 6

### Determination of Barrier Function of the Skin

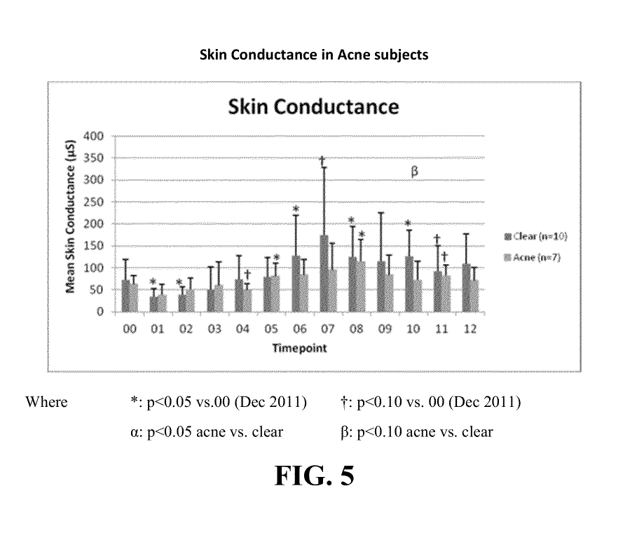

The barrier function of the skin was measured by determining the transepidermal water loss (TEWL) and the skin conductance using the methods of Assay 2 and 3 respectively. The results are respectively plotted in graphs 4 and 5, which graphs are respectively depicted in FIGS. 4 and 5.

As it can be seen from Graph 4, the levels of TEWL are significantly higher in the acne group when compared to the Clear one throughout a period of 12 consecutive months of once per month measurements (00-12 months). The observed fluctuation in in the levels correlates with seasonal changes in both groups. Graph 5 shows higher Skin Conductance levels in the Clear Group with higher difference observed in the summer months of 06-09.

Ceramides are lipid components of the skin which are an important part of the outer layer of skin, and therefore important in protecting the barrier function of skin. The correlation of ceramide levels with the severity of acne progression is demonstrated in example 8, with higher levels of ceramides in skin showing lower acne grading. The impairment of barrier function in acne subjects is demonstrated in example 5 with skin of acne subjects having higher TEWL values and lower skin conductance than the subjects with clear skin. The preceding examples 6-7 demonstrate the ability of non-polar extracts of Malva neglecta (E1) to induce expression of ceramide synthesis and transport genes, as well as functionally to increase the endogenous production of ceramides.

Collectively, these changes indicate that non-polar extracts of Malva neglecta (E1) have the ability to induce physiological changes that positively affect skin barrier function and improve the appearance of at least one sign of acne. By increasing the production of ceramides and skin lipids, Malva neglecta extracts would be expected to regain the ratio of ceramides:free fatty acids:cholesterol and, thus, strengthen the barrier of skin thereby resulting in improving the appearance of at least one sign of acne.

What is claimed is:

1. A composition of a non-polar, or lipophilic, or non-polar lipophilic extract of Malva neglecta, cholesterol, and a cosmetically acceptable topical carrier.

2. A composition as in claim 1, further comprising an active agent for reducing at least one sign of acne.

3. A composition as in claim 1, wherein said topical composition comprises from about 0.001% to about 90% of an extract of Malva neglecta.

4. A composition as in claim 1, wherein said topical composition comprises from about 0.01% to about 20% by weight of said extract of Malva neglecta herb.

5. A composition as in claim 1, wherein said topical composition comprises about 0.01 to about 5% of said extract of Malva neglecta.

6. A composition as in claim 1, wherein said composition comprises from about 0.01% to about 2% of said extract of Malva neglecta.

7. A composition as in claim 1, further comprising an additional ceramide-production-inducing agent.

8. A composition as in claim 7, wherein said ceramide-production-inducing agent is Bursera simaruba.

9. A composition as in claim 8, wherein said ceramide-production-inducing agent is an extract of Bursera simaruba seeds.

10. A composition as in claim 7, wherein said ceramide-production-inducing agent is present in an amount sufficient to induce production of ceramides.

11. A composition as in claim 1, wherein said composition further comprises a free fatty acid.

12. A composition as in claim 1, wherein said extract comprises non-polar, or lipophilic, or non-polar lipophilic extract of Malva neglecta.

13. A composition as in claim 12, wherein said extract is a non-polar extract.

14. A composition as in claim 13, wherein said extract is essentially free of polar components.

15. A composition as in claim 1, wherein said extract is a hexane extract.
